SEQUENCE LISTING (10 sequences)

SEQ ID NO: 1 - protein, 21 aa, Artificial Sequence
  Description of Artificial Sequence Synthetic peptide
MKQLADSLHQLARQVRSLEHA

SEQ ID NO: 2 - DNA, 97 bp, Artificial Sequence
  Description of Artificial Sequence Synthetic oligonucleotide
tcagaccaactctattacctccctgtataaatctgcgggcagcgaaaacctgtacttccaacagcaacgcttccagtggcaatttgaacagcaataa

SEQ ID NO: 3 - DNA, 97 bp, Artificial Sequence
  Description of Artificial Sequence Synthetic oligonucleotide
ttattgctgttcaaattgccactggaagcgttgctgttggaagtacaggttttcgctgcccgcagatttatacagggaggtaatagagttggtctga

SEQ ID NO: 4 - protein, 14 aa, Artificial Sequence
  Description of Artificial Sequence Synthetic peptide
QTNSITSLYKSAGS

SEQ ID NO: 5 - protein, 6 aa, Artificial Sequence
  Description of Artificial Sequence Synthetic peptide
ENLYFQ

SEQ ID NO: 6 - protein, 11 aa, Artificial Sequence
  Description of Artificial Sequence Synthetic peptide
QQRFQWQFEQQ

SEQ ID NO: 7 - protein, 6 aa, Artificial Sequence
  Description of Artificial Sequence Synthetic 6xHis tag
HHHHHH

SEQ ID NO: 8 - protein, 5 aa, Artificial Sequence
  Description of Artificial Sequence Synthetic peptide
DDDDK

SEQ ID NO: 9 - protein, 7 aa, Artificial Sequence
  Description of Artificial Sequence Synthetic peptide
ENLYFQG

SEQ ID NO: 10 - protein, 16 aa, Artificial Sequence
  Description of Artificial Sequence Synthetic peptide
ENLYFQQQRFWQFEQQ

(19) United States
(12) Patent Application Publication
(10) Pub. No.: US 2010/0273983 A1
(43) Pub. Date: Oct. 28, 2010

(54) METHOD OF PURIFYING PEPTIDES BY SELECTIVE PRECIPITATION

(75) Inventors: Waltraud Kaar, Indooroopilly (AU); Robert John Falconer, Auchenflower (AU); Anton Peter Jacob Middelberg, Brookfield (AU); Belinda Maree Hartmann, Auchenflower (AU)

Correspondence Address: EDWARDS ANGELL PALMER & DODGE LLP, P.O. BOX 55874, BOSTON, MA, 02205, US

(73) Assignee: The University of Queensland, Brisbane (AU)

(21) Appl. No.: 12/594,821

(22) PCT Filed: Apr. 4, 2008
(86) PCT No.: PCT/AU08/00497

§ 371 Date: Feb. 25, 2010

(30) Foreign Application Priority Data
Apr. 5, 2007 (AU) 2007901829

(51) Int. Cl. C07K 1/32; C07K 14/00

(52) U.S. Cl. 530/326; 530/344

(57) ABSTRACT

A method is provided for purifying peptides by selective precipitation of contaminating proteins, such as host cell proteins and cleaved fusion partners. Also provided is a method of cleaving fusion proteins in cell lysates.

### FIELD OF THE INVENTION

The present invention relates to methods for purifying recombinant peptides. In particular, the invention relates to a method for purifying a recombinant peptide from its fusion partner. This invention also relates to peptides obtained by these methods.

### BACKGROUND TO THE INVENTION

A growing interest in peptides has developed as a result of their often unique biological and self-assembly characteristics. Peptides are increasingly being exploited for the development of new bioactives and biomaterials such as filaments and fibrils, hydrogels, surfactants, peptide hybrids, hormones and antibiotics and therapeutic agents.

Traditionally, peptides have been produced by stepwise solid phase chemical synthesis using techniques based on that devised by Merrifield. Chemical synthesis is a rapid and effective method for the production of custom-made peptides in small quantities. However, chemical synthesis methods have major drawbacks in that they are often prohibitively expensive; and only relatively small quantities can be produced per synthesis. The cost of peptides made by this method is typically between $300 and $500 per g for 300 to 500 g quantities; between $100 and $200 per g for 1 to 2 kg quantities; from $25 to $50 per g at the 50 to 100 kg scale, and in the order of $10 per g at greater scales. Since synthesis involves a series of deprotecting, linking, washing, re-deprotecting, relinking, and rewashing steps the costs of chemical synthesis increase with peptide chain length and sequences of over 35 amino acids are not generally economically feasible. In addition, the process employs toxic chemicals that present sustainability problems.

In contrast, recombinant peptide production in micro-organisms such as bacteria, has the potential to avoid some of the toxicity and quantity issues encountered with chemical synthesis. This technology can be extended to peptide expression to dramatically decrease costs compared with those from chemical synthesis. By definition, peptides are small, short sequences, which are either poorly expressed in E. coli, or are rapidly degraded. It is for this reason that they are produced as a part of a much larger fusion protein, with the target peptide only released during downstream processing after cleavage at a strategically located position in the amino acid sequence. The fusion partner can also be engineered to facilitate purification, detection of fusion protein from a complex mixture, or to avoid degradation by endogenous host proteases.

Recovery of polypeptides from recombinant protein expression systems typically involves multi-step purification schemes. Therefore, efficient and cost-effective is downstream processing techniques are essential for handling the large volumes produced by fermentation. However, the traditional route for recombinant protein recovery involves a large number of costly and inefficient purification steps. Cells are disrupted by high-pressure homogenisation and the insoluble inclusion bodies are collected by centrifugation. Inclusion bodies are then solubilised using a strong denaturant prior to refolding. Multiple cell disruption passes are required to decrease cell debris size. Subsequent purification is achieved by batch column, ion-exchange, size exclusion, reversed phase, hydrophobic interaction, or affinity chromatography. These traditional chromatography steps are time-consuming, expensive and not readily adaptable to large scale production processes. In addition, the type of chromatography applicable in each case must be adjusted based on the characteristics of the particular peptide of interest.

A further disadvantage arising from the use of fusion proteins is that liberation of the fusion partner from the peptide by chemical or enzymatic cleavage, and subsequent purification of the peptide from the fusion partner and other contaminants, can be difficult and expensive, and the resulting yield of the peptide is greatly diminished, since the peptide represents only a fraction of the entire fusion protein.

Accordingly, there is a need for improved methods for purifying recombinant peptides that address the above problems.

### SUMMARY OF THE INVENTION

We have now found that the traditional chromatography steps carried out during peptide purification can be replaced by steps based on preferential precipitation of contaminating proteins, including host proteins and cleaved fusion partners, leaving the peptide in solution for subsequent recovery.

Accordingly in a first aspect the present invention provides a method of purifying a recombinant peptide which method comprises (i) providing a solution comprising a recombinant peptide and a polypeptide fusion partner; (ii) subjecting the solution to conditions, wherein the fusion partner is preferentially precipitated; and (iii) separating the resulting precipitate from the solution.

In addition, we have found that it is feasible to carry out the cleavage of the fusion polypeptide in the cell homogenate/lysate without the need for a chromatographic purification step prior to the cleavage step. This further eliminates expensive chromatography/purification steps—the first lysate-cleanup stage is typically the most costly because of the high lysate volume and low initial purity.

Accordingly, in a particularly preferred embodiment, the solution in step (i) is a cell lysate or a partially purified derivative thereof comprising host cell proteins. Thus, in a related aspect, the present invention provides a method of purifying a recombinant peptide, which method comprises (i) providing a cell lysate or a partially purified derivative thereof comprising host cell proteins, such as a cell lysate supernatant, which cell lysate or derivative thereof comprises a recombinant peptide and a polypeptide fusion partner; (ii) subjecting the cell lysate or derivative thereof to conditions wherein the fusion partner and host cell proteins are preferentially precipitated; and (iii) separating the resulting precipitate from the recombinant peptide remaining in solution.

In a further related aspect, the present invention provides a method of purifying a recombinant polypeptide, which method comprises

(i) providing a cell lysate or a partially purified derivative thereof comprising a recombinant fusion protein comprising a polypeptide of interest fused to a polypeptide fusion partner;
(ii) optionally treating the cell lysate or partially purified derivative thereof to inactive host cell protease activity present in the cell lysate or a partially purified derivative thereof, e.g. a heat treatment step;
(iii) adding a cleavage agent which cleaves the polypeptide of interest from the polypeptide fusion partner; and
(iv) subjecting the cell lysate or derivative thereof to conditions wherein the fusion partner and host cell proteins are preferentially precipitated; and
(v) separating the resulting precipitate from the recombinant peptide remaining in solution.

In one aspect, it is preferred that purification of the recombinant polypeptide/peptide of interest, from host cell expression through to final purification of the recombinant polypeptide/peptide does not include any chromatographic steps. Thus in one aspect the invention relates to a non-chromatographic method of purifying recombinant polypeptides of interest, which have been expressed as fusion proteins in a host cell, which method comprises cleaving the polypeptide of interest from its fusion partner in the host cell lysate/homogenate and then isolating the polypeptide of interest from its fusion partner and host cell proteins present in the host cell lysate/homogenate.

It is preferred that the in-lysate digestion/cleavage step referred to in the various aspects above is accompanied by an initial treatment step to substantially reduce or eliminate host cell proteases, e.g. a heat treatment step.

This second improvement to the purification of recombinant proteins (i.e. in-lysate cleavage) is more generally applicable and need not be followed by a selective precipitation step. The subsequent purification can be accomplished by other means, such as selective adsorption methods to selectively capture the cleavage recombinant polypeptide of interest.

Accordingly, in another aspect, the present invention provides a method of purifying a recombinant polypeptide which method comprises the steps of:

(i) providing a cell lysate or a partially purified derivative thereof comprising a recombinant fusion protein comprising a polypeptide of interest fused to a polypeptide fusion partner;
(ii) optionally treating the cell lysate or partially purified derivative thereof to to inactive host cell protease activity present in the cell lysate or a partially purified derivative thereof, e.g. a heat treatment step;
(iii) adding a cleavage agent which cleaves the polypeptide of interest from the polypeptide fusion partner; and
(iv) isolating the polypeptide of interest from the polypeptide fusion partner and any host cell proteins present in the cell lysate or partially purified derivative thereof.

In one embodiment of the various aspects of the invention described above the conditions wherein the fusion partner and host cell proteins are preferentially precipitated comprise adding a non-aqueous polar solvent to the solution. Preferably, the non-aqueous polar solvent is added to a concentration of at least 30% v/v, more preferably at least 45% v/v or 60% v/v.

In one embodiment the non-aqueous polar solvent is an aliphatic alcohol, such as ethanol and/or an aliphatic ketone, such as acetone.

Preferably, the recombinant peptide comprises from 10 to 70 amino acids, more preferably 50 amino acids or less, such as from 10 to 30 amino acids.

The present invention also provides a polypeptide or peptide which is obtained or obtainable by the method of the invention.

### DETAILED DESCRIPTION OF THE INVENTION

Unless defined otherwise, all technical and scientific terms used herein have the same meaning as commonly understood by one of ordinary skill in the art (e.g. in cell biology, chemistry and molecular biology). Standard techniques used for molecular and biochemical methods can be found in Sambrook et al., Molecular Cloning: A Laboratory Manual, 3^{rd} ed. (2001) Cold Spring Harbor Laboratory Press, Cold Spring Harbor, N.Y. and Ausubel et al., Short Protocols in Molecular Biology (1999) 4^{th} Ed, John Wiley & Sons, Inc.—and the full version entitled Current Protocols in Molecular Biology).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, reference to numerical values, unless stated otherwise, is to be taken as meaning “about” that numerical value. The term “about” is used to indicate that a value includes the inherent variation of error for the device and the method being employed to determine the value, or the variation that exists among the study subjects.

Temperature values are given for conditions of standard pressure (1 atm).

### Recombinant Peptides

The term “peptide” as used herein means a polymer made up of two or more amino acids linked together by peptide bonds. The term “recombinant peptide” as described herein refers to a peptide which is produced by recombinant methods. The maximum length for recombinant peptides in the context of the present invention is generally selected such that the peptides are shorter than the polypeptide fusion partners from which it is desired to separate them (e.g. maltose binding protein has about 400 amino acids, glutathione S-transferase (GST) has about 200 amino acids), preferably at least 100 or 150 amino acids shorter than the polypeptide fusion partner.

Typically the recombinant peptide is between 10 and 100 amino acids in length. Preferably the recombinant peptide is less than or equal to 70 or 50 amino acids in length, for example, the peptide may have from 10 to 30 amino acids.

The term “amino acid” as used herein encompasses both natural and non-naturally occurring amino acids, the latter generally either being capable of being incorporated during recombinant protein synthesis or resulting from post-translational modification.

Particular recombinant peptides of interest include therapeutic peptides, peptide surfactants, for example the 21 residue peptide AM1 described herein, that acts is as a stimuli responsive peptide, and peptide hormones, antigens or mutant peptides produced by protein engineering techniques, or synthetic peptides (e.g. designed peptides). Other specific examples include P₁₁ peptides-glutamine rich, β-sheet forming peptides (Aggeli et al, 1997, Nature 386(6622):259-62.; Aggeli et al., 2001, PNAS 98(21):11857-62, Aggeli et al., 2003, Journal of the American Chemical Society 125(32):9619-28)—e.g. the P₁₁-2 peptide described herein.

The recombinant peptides are generally produced as fusion proteins, in frame with a larger polypeptide termed a fusion partner, expressed in a host cell from a vector or similar molecule. The host cells may be prokaryotic, e.g. bacteria such as E. coli, or eukaryotic, e.g. plant cells, fungal cells such as yeast or filamentous fungi. Examples of yeast include Saccharomyces sp. (e.g. S. cerevisiae), Pichia sp. (e.g. P. pastoris) and Kluyveromyces sp. (e.g. K. lactis). Expression in recombinant plants may occur in a tissue-specific manner, such as in seed, fruit or leaf tissue. Examples of suitable plants include oil seed-bearing plants, cereals, legumes and fruit, vegetable and leafy salad crops. Particular plant species that may be suitable for recombinant peptide production include Triticum species such as Triticum aestivum, the Brassicaceae such as Brassica rapa, Brasica campestris L., Brassica napus L., cotton (Gossypium hirsutum L.), maize (Zea mays), rice (Oryza sativa), potato (Solanum tuberosum L), yam (the Dioscoreaceae), cassaya (Manihot esculentum Crantz), sugar beet (Beta vulgaris), linseed (Linum sp.), and various forage and fodder crops such as clovers and grasses. See also Ma et al., 2003, Nature Review Genetics 4(10): 794-805.

The recombinant peptide of the invention may also be produced as part of a biorefinery, for example a whole-crop biorefinery, green biorefinery or lignocellulose-feedstock biorefinery

Recombinant production of peptides as fusion proteins can be achieved using various techniques. Typically the polynucleotide sequence of interest is cloned into an “expression vector”. The vector may be a plasmid vector, a viral vector, or any other suitable vehicle adapted for the insertion of foreign sequences, their introduction into eukaryotic or prokaryotic cells and the expression of the introduced sequences as appropriate. Typically the vector includes transcriptional/translational control sequences required for expression of the fusion protein in a host cell, such as a promoter, a ribosome binding site, an initiation codon, a stop codon, optionally an operator sequence and possibly other regulatory sequences such as enhancers.

The recombinant expression vectors suitable for producing the recombinant peptides of the invention typically include one or more regulatory sequences, selected on the basis of the host cells to be used for expression, operably linked to the nucleic acid sequence encoding the fusion protein to be expressed. It will be appreciated by those skilled in the art that the design of the expression vector can depend on such factors as the choice of the host cell to be transformed and the level of expression of peptide desired. Vector DNA can be introduced into prokaryotic or eukaryotic cells via conventional transformation or transfection techniques. Methods and materials for preparing recombinant vectors, transforming host cells using replicating vectors, and expressing biologically active foreign polypeptides and peptides are generally well known in the art.

In one embodiment the recombinant peptides/fusion proteins are produced in a substantially soluble form. In an alternative embodiment, the recombinant peptides/fusion proteins are produced in an insoluble state. If the recombinant peptide/fusion protein is expressed in an insoluble state, e.g. it accumulates in insoluble inclusion bodies in the host cell, it is subsequently solubilised by methods known to a person of skill in the art, e.g. using chaotropic agents such as urea, before the precipitation steps of the method of the invention are performed.

The recombinant peptides of the invention may also be produced in a cell-free system.

The recombinant peptides of the invention may also be designed so that they are secreted extracellularly and/or transported to specific locations in the cell. For example, the recombinant peptide may be designed to be transported to an organelle, such as plastid or vacuole in a plant cell or other type of non-membrane cellular body or inclusion such as those that exist in prokaryotes. Methods employed to target peptides to extra-cellular or cellular locations are generally known in the art. For example, recombinant peptides and/or vector constructs may be designed to include targeting sequence(s) and/or post-translation modifications that enable cellular transport. Such transport may be post-translational or co-translational.

The recombinant peptides may also be produced in a particular organelle or cellular location, such as a plastid. Appropriate methods for achieving expression in the desired location are known to those of skill in the art and involve, for example design of the recombinant peptide and/or vectors with suitable promoters and other 5′ and 3′ control sequences as required.

In one embodiment, the recombinant peptide (as a separate peptide from any fusion partner) has an isoelectric point, or pI, of around the average host protein or lower. In an alternative embodiment the recombinant peptide has a pI of around the average host protein or higher. Such average pI can be determined by methods such as 2D electrophoresis, or isoelectrofocusing alone or chromatofocusing or calculations based on amino acid composition.

### Recombinant Polypeptides of Interest

In another aspect of the invention, the purification process does not involve a selective precipitation step (i.e. where the in-lysate cleavage step has been used). In this aspect, there is no particular size limitation to the polypeptide of interest. Thus, a recombinant polypeptide of interest may have more than 100 amino acids, by contrast to the shorter peptides referred to above. In all other respects, the disclosure above in relation to “peptides” is equally applicable as appropriate to polypeptides in general, of which peptides can be considered a subset.

### Fusion Partner Polypeptides

Recombinant polypeptides/peptides of interest are generally expressed together with a polypeptide fusion partner as a fusion protein. This is because recombinant peptides are usually either poorly expressed in host cells, or are rapidly degraded. In this case, the recombinant peptide is produced by the host cell, together with the fusion partner, and the recombinant peptide of interest is then released during downstream processing after cleavage at a particular site in the amino acid sequence, such as a protease recognition site.

There are various fusion partners available in the art with different properties suitable for the present application. It is to be understood that the fusion partner may be selected to suit each particular peptide in relation to a desired end use or effect. The fusion partner can be engineered to facilitate purification, detection of fusion protein from a complex mixture, and/or to avoid degradation by endogenous host proteases. The fusion partner may also facilitate or allow transport of the recombinant peptide to extracellular or intracellular locations, such as a particular organelle (e.g. plastids such as chloroplasts and amyloplasts), i.e. the fusion partner may comprise a targeting sequence which either directs the fusion protein to be secreted or to particular cellular compartments/organelles. Various targeting sequences are known in the art.

Examples of fusion partners include maltose binding protein (MBP), such as encoded by the malE gene in E. coli, and glutathione S-transferase (GST), thioredoxin, NUS A, ubiquitin (Ub) and small ubiquitin-related modifier (SUMO) (Marblestone et al., 2006, Protein Science 15: 182-9). In one embodiment (where a selective precipitation step is used in accordance with the present invention), preferred fusion partners have at least 60, 70, 80, 90, 100 or 150 amino acids, or at least 50, 70 or 100 more amino acids than the peptide of interest (NUS A has about 495 amino acids; MBP has about 400 amino acids; GST has about 200 amino acids; thioredoxin has about 109 amino acids; SUMO has about 100 amino acids and ubiquitin has about 76 amino acids). In a particular embodiment, carbonic anhydrase and fragments thereof are specifically is excluded as fusion partners in the context of the present invention.

In one embodiment, smaller fusion partners are preferred for economic reasons since the ratio of recombinant peptide to fusion partner is higher (e.g. at least 1:20, such as at least 1:15 or 1:10) and therefore the yield of recombinant peptide in relation to total recombinant fusion protein is higher. Thus in this embodiment, the fusion partner has less than 200 amino acids, such as less than 150 amino acids.

In another embodiment, where selective precipitation is not used to purify the cleaved polypeptide of interest from its fusion partner, the fusion partner may be much smaller, such as a hexahistidine tag or a myc epitope tag. In this embodiment, the fusion partner may therefore be less than 50 amino acids but preferably at least 6 or 10 amino acids. Preferably, the combined size of the fusion partner and the polypeptide of interest are such that the expressed fusion protein is stable in the host cell and not subject to significant degradation.

The fusion partner preferably increases the levels of expression and/or stability of the recombinant peptide to which it is fused. In one embodiment the fusion partner increases the solubility of the recombinant peptide to which it is fused.

In one embodiment, the fusion protein includes a spacer between the fusion partner and the (poly)peptide of interest. If present, the spacer may simply comprise one or more, e.g. three to ten amino acid residues, separating the fusion partner from the peptide of interest. In some embodiments, the spacer may comprise a proteolytic cleavage site, as discussed below.

The fusion protein preferably comprises a cleavage site to enable release of the (poly)peptide of interest from the fusion partner by proteolytic cleavage by a protease (i.e. the cleavage site includes a consensus recognition sequence for a site-specific protease).

The terms “cleavage site” used herein refers to an amino acid sequence which is recognized and cleaved by an enzyme or chemical means at the scissile bond. The term “scissile bond” referred to herein is the juncture where cleavage occurs; for example the scissile bond recognized by enterokinase may be the bond following the sequence (Asp4)-Lys in the spacer peptide or affinity peptide.

Examples of proteases useful for cleavage of fusion partners from peptides of interest include factor Xa, papain, pepsin, plasmin, thrombin, enterokinase, Tobacco Etch Virus (TEV) protease and the like. Each effects cleavage at a particular amino acid sequence which it recognizes (the cleavage site). It is preferred that the protease is selected such that there is only one cleavage site in the fusion protein or such that the fusion protein is cleaved into polypeptide fragments that are still sufficiently large to enable them to be preferentially precipitated (e.g. at least 60, 80, 100 or 150 amino acids in length).

In certain embodiments, digestion with the cleavage enzyme may occur, for example, while the peptide is still in the crude cell lysate or lysate supernatant or after further purification steps have been carried out. After cleavage with the proteolytic agent, an endopeptidase such as trypsin, clostropain or furin may also be used, where appropriate, to remove any remaining amino acids residues in the spacer region that are not desired as part of the recombinant peptide.

### Preferential Precipitation of Recombinant Peptides of Interest

In one aspect, the present invention is based on the preferential precipitation of contaminating proteins from a solution containing a recombinant peptide of interest i.e. all or most of the contaminating proteins are precipitated whilst all or most of the peptide is retained in solution. Thus a solution comprising both the peptide of interest and soluble contaminating proteins is subjected to conditions which result in preferential precipitation of the contaminating proteins (e.g. host cell proteins, cleaved polypeptide fusion partner and proteinaceous cleavage agents such as proteases). The precipitate can then be separated from the solution containing the peptide of interest which can then be recovered from that solution by suitable techniques such as freeze drying etc.

The term “precipitation” refers to the formation of an insoluble solid by a reaction which occurs in solution. For example, precipitation can occur upon addition of a suitable precipitation agent to a solution. When precipitation occurs, the solid formed as a result is called the “precipitate”. The precipitate can be collected or separated from the remaining solution by various methods, such as filtration, decanting, centrifuging and the like.

Typically in the context of the present invention, precipitation occurs as a result of the addition of a precipitating agent to the solution containing the recombinant peptide of interest and the contaminating proteins (e.g. host cell proteins, cleaved polypeptide fusion partner and proteinaceous cleavage agents such as proteases). Thus in one embodiment, subjecting the solution to conditions wherein contaminating proteins such as a cleaved fusion partners are preferentially precipitated includes the step of adding to the solution a precipitating agent.

The precipitating agent is added to the solution in an amount sufficient to provide conditions that result in preferential precipitation of the contaminating protein material from solution whilst leaving the peptide(s) of interest in solution. Preferably at least 80, 90, 95, 98 or 99% of the contaminating protein material is precipitated. Preferably at least 50, 60, 70, 80 or 90% of the peptide(s) of interest remain in solution.

In one embodiment, the precipitating agent is an agent which is capable of causing the contaminating proteins to denature and aggregate, thus causing them to precipitate. Denaturation and aggregation can also be achieved by heating the solution to an elevated temperature. The required temperature will vary between different proteins, and can also depend on other factors such as the presence of precipitating agents, including agents that alter the pH of the solution (typically a reduction or increase in pH from normal physiological pH means that smaller increases in temperature will give rise to the desired selective precipitation). In one embodiment, the solution is maintained during the precipitation step at a temperature of at least 10° C., such as at least 15 or 20° C., typically less than 37 or 30° C.

In a preferred embodiment the precipitating agent is a non-aqueous polar solvent. The term “non-aqueous solvent” means a solvent other than water. The term “polar” as used herein relates to the overall polarity of a particular compound. A polar molecule usually contains polar bonds, which have unequal sharing of electrons between the two atoms involved in bonding. A non-polar compound usually contains non-polar bonds, which have identical or similar sharing of electrons. As is known in the art, besides bond polarity, the other factor that decides if a molecule is polar is the symmetry of a molecule. Whilst molecules can be described as “polar” it must be noted that this is often a relative term, with one molecule simply being more polar or more non-polar than another. As such, as is known in the art there are no absolute properties which can be ascribed to polar molecules. However, the following properties are typical of such molecules. Polar molecules are generally hydrophilic and are molecules that usually have slightly positive and slightly negatively charged ends. Typically, the non-aqueous polar solvent is miscible with water.

In one embodiment the precipitating agent is an alcohol that includes one or more carbon atoms, and at least one hydroxyl group. The alcohol may be an aliphatic alcohol, such as a primary, secondary or tertiary aliphatic alcohol. For example, the alcohol may be monohydroxy alcohols such as ethanol, methanol, isopropanol or n-propanol; diols or triols such as ethylene glycol, propylene glycol, glycerol or 1,2-hexanediol; or polyglycols such as polyethylene glycol. In a preferred embodiment the precipitating agent is ethanol.

In an alternative embodiment, the precipitating agent is not an alcohol. For example non-aqueous polar solvents such as an aprotic polar solvent (e.g. aliphatic ketones, such as acetone), dioxane, dimethylsulphoxide, dimethylformamide and acetonitrile are also suitable precipitating agents.

Precipitation can also be achieved by altering the pH. For example the pH can be decreased to less than 5 or increased to more than 9. The optimum pH will vary between different fusion partners/host cell proteins. In an alternative embodiment, precipitation takes place at around physiological pH, e.g. at from pH 6.5 to 8.0.

Preferably the precipitating agent is relatively inert with respect to the peptide, such that the precipitating agent does not chemically react with the peptide and/or other moieties present in the composition.

The amount of precipitating agent added to the solution is chosen to take into account the need to preferentially precipitate contaminating proteins whilst retaining a substantial proportion of the peptide of interest in solution. In one embodiment, the precipitating agent added to give a final amount of at least 10% v/v such as at least 20, 25 or 30% v/v. In a preferred embodiment, particularly where the precipitating agent is a non-aqueous polar solvent such as an alcohol, the precipitating agent is added to give a final amount of at least 40, 45, 50, 55 or 60% v/v.

Typically, the precipitating agent is added to give a final amount of 90% v/v or less, such as 85, 80, 75, 70% v/v or less. For example a precipitating agent, such as a non-aqueous polar solvent, may be added to a final concentration from about 45% v/v to 75% v/v, such as from 60% v/v to 75% v/v.

Combinations of precipitating agents may also be used and in which case the amounts given above refer to the total amount of all of the precipitating agents.

In a preferred embodiment, the precipitation conditions include the presence of at least 100 mM salt (e.g NaCl), such as at least 150, 200 or 250 mM salt, but generally less than 1M salt, such as less than 750 mM or 500 mM salt. The salt is included to reduce aggregation of the recombinant peptide with other proteins.

The inclusion of salt is particularly preferred where a non-aqueous polar solvent is used as a precipitation agent.

Following precipitating, the resulting solution is typically processed further to separate the precipitate from the dissolved peptides of interest. Suitable techniques include centrifugation and filtration.

### Applications

The present invention may be used to purify any (poly)peptide that can be expressed as the product of recombinant technology in a host cell. These recombinant (poly)peptide products include therapeutic (poly)peptides, surfactants, peptide hormones, mutant peptides produced by protein engineering techniques, and synthetic peptides (e.g. designer peptides).

The term “purified” means that the molecule in question has been removed from its natural environment or host, and associated impurities reduced or removed such that the molecule in question is the predominant species present (e.g., on a molar basis it is more abundant than any other individual species in the composition/solution). Typically, a composition comprising purified recombinant (poly)peptide(s) of interest is one where the peptide(s) of interest represent at least 30 percent w/w of all macromolecular species present, preferably at least 50, 60, 70 or 75% w/w. A substantially pure composition will comprise more than 80 to 90% w/w of recombinant peptide(s) of interest.

The solution containing the peptide of interest from which the contaminating proteins are precipitated may be derived from a lysate/homogenate of a culture of host cells, such as microbial or fungal host cells, expressing a fusion protein which includes the peptide of interest and a fusion partner. Procedures for the expression of recombinant peptides, on the desired scale, are known in the art.

Typically, after expression has been achieved to a desired level or length of time, the cells can by lysed and the fusion protein solubilised if not already in soluble is form, by conventional techniques, such as sonication, enzymatic digestion, mechanical homogenisation and the like. Once lysis/solubilisation has been completed, coarse cellular debris can optionally be removed by, for example, centrifugation or filtration and the like. The cell lysate may also be subject to a volume reduction step.

Another optional purification step is the purification of fusion protein from other host cell contaminants by a chromatographic step, such as affinity chromatography based on the fusion partner (alternatively, it is possible to have two different fusion partners, one to assist with expression of the peptide and another which may be a fairly short fusion partner (e.g. hexahistidine) to assist with affinity purification. Where the affinity tag is short, the two fusion partners will generally be fused to one another rather than flanking the peptide of interest so that the short affinity tag can be cleaved as part of a larger polypeptide). However, in some embodiments, it is preferred that the fusion protein is not separated from the host cell contaminants by chromatography due to the additional cost and complexity involved.

A “partially purified derivative of a cell lysate” as referred to herein is therefore the solution/composition resulting from partial purification of the host cell lysate e.g. a cell lysate supernatant. Such a derivative may comprise host cell proteins or may have been subject to purification steps (preferably non-chromatographic) to separate some or all of the host cell protein from the recombinant fusion protein.

In one embodiment, the cell lysate or partially purified derivative thereof is treated, e.g. heat treated, to inactivate some or all host cell proteins, such as proteases. Typically, the cell lysate or partially purified derivative thereof is heated to a temperature of from 45 to 70° C., such as at least 50° C., preferably less than 60° C. The temperature should be chosen so as to avoid precipitation of the recombinant peptide of interest.

The cell lysate or partially purified derivative thereof, can be adjusted, if necessary, to provide conditions suitable for the cleavage or removal of the polypeptide fusion partner from the recombinant peptide. Various methods for the cleavage of fusion partners from recombinant peptides is known in the art. As mentioned above, typically the cleavage of the fusion partner is carried out by a suitable protease.

Thus the host cell lysate may be subject to a number of steps, some of which are optional, to produce a solution comprising polypeptide fusion partner and cleaved peptide of interest. Depending on the steps carried out, the solution may also comprise host cell proteins and/or the cleavage agent, such as a protease, used to cleave the fusion partner from the peptide of interest.

Alternatively, the fusion protein may be present in the culture medium as a result of secretion from the host cell. In this case, optional purification steps may be carried out to partially purify (e.g. to remove host cells and host cell debris) and/or concentrate the fusion protein present in the culture medium to obtain a solution comprising the fusion protein.

In another embodiment, where the fusion protein is expressed in a transgenic plant, initial treatment steps will typically be required to extract the fusion protein from the plant or part thereof, such as mechanical and/or enzymatic extraction. In many cases, recombinant proteins can be extracted by simply adding an aqueous buffer to ground tissue. The pH and salt content can be optimized on a case by case basis to reduce extraction of endogenous proteins and preferentially solubilise the recombinant protein (Evangelista et al., 1998; Process and economic evaluation of the extraction and purification of recombinant β-glucuronidase from transgenic corn. Biotechnol. Prog. 14: 607-614; Bai and Nikolov, 2001, Effect of processing on the recovery of recombinant β-glucuronidase (Gus) from transgenic canola. Biotechnol. Prog. 17: 168-174).

The recombinant peptide present in the solution can then be purified, as described in detail above, by subjecting the solution to conditions whereby the contaminating proteins, such as the fusion partner and cleavage enzyme are preferentially precipitated.

The soluble peptide fraction may then be separated from the precipitate using techniques such as centrifugation or filtration.

Alternatively, where an in-lysate cleavage step has been performed, the recombinant polypeptide/peptide can be separated from the fusion protein and host cell proteins using other purification steps, such as selective adsorption, e.g. using magnetic nanoparticles.

The solution may be treated to remove some or all of the solvent e.g. by freeze drying, spray-drying, lyophilisation, or to otherwise recover the recombinant peptide from the solution. The recombinant peptide may then be stored, for example, as a liquid formulation or solid preparation. The desired formulation of the end product will be determined by the required downstream application of the peptide

The described methods may be applied to any scale of peptide production. For example, the method may be applied to relatively small scale expression cultures or may be suitably applied to the large scale production of peptides from cultures of host cells in industrial scale fermenters or bioreactors (e.g. with culture volumes of 20, 50 or more litres). The methods of the present invention can be used in a batch-wise manner or, for example, in a continuously run or automated system.

The present invention will now be further described with reference to the following examples, which are illustrative only and non-limiting. The examples refer to figures:

### DESCRIPTION OF THE FIGURES

FIG. 1 is a graph showing the results of a HPLC analysis of the presence of AM1 peptide in the supernatants and pellets obtained from spiked homogenates precipitated with different ethanol concentrations Included on this graph are the corresponding Bradford analysis results of total protein in the same supernatants and pellets. Both sets of data are expressed as percentages of the AM1 or total protein present in the starting material. Incubation temperature: 23° C., incubation time: 17 h, mixing on roller shaker, centrifugation: 10 min at 15,000 rpm. Ethanol concentration is expressed as percent weight/weight.

FIG. 2 is a representation of the Trx-P₁₁-2 expression product comprising (i) spacer, (ii) TEVp consensus sequence, (iii) P₁₁-2 peptide sequence and (1) site of cleavage.

FIG. 3 is a representation of the solubility of protein and peptide in Trx-P₁₁-2 homogenate following TEVp cleavage and precipitation with ethanol and acetone.
A) Graph showing peptide and protein recovery in the pellet and the supernatant derived from precipitation of TEVp cleaved Trx-P₁₁-2 homogenate with increasing levels of ethanol (left y-axis) and total recovery (pellet+supernatant) (right y-axis) based on quantification of peptide and protein in starting material.
B) Graph showing peptide and protein recovery in the pellet and the supernatant derived from precipitation of TEVp cleaved Trx-P₁₁-2 homogenate with increasing levels of acetone (left y-axis) and total recovery (pellet+supernatant) (right y-axis) based on quantification of peptide and protein in starting material.

FIG. 4 depicts the Mass spectrum analysis of supernatant generated following cleavage and precipitation (75% v/v ethanol) of the Trx-P₁₁-2 homogenate. The P₁₁-2 peptide elutes at 1551 daltons.

FIG. 5 is a flow diagram depicting one embodiment of the process of the invention.

### EXAMPLES

### Example 1

AM1 is a designed 21-residue peptide (Ac-MKQLADSLHQLARQVRSLEHA-CONH₂) (SEQ ID NO: 1), which is a stimuli-responsive surfactant capable of converting between a mechanically strong and cohesive ‘film state’ and a mobile ‘detergent state’ at a fluid-fluid interface in response to changes in bulk aqueous solution composition (Dexter, et al., 2006; Reversible active switching of the mechanical properties of a peptide film at a fluid-fluid interface. Nature Materials 5 (6): 502-506). A model was designed to investigate the purification of a peptide AM1 after it has been cleaved off from the fusion partner maltose-binding protein (MBP). In this model, chemically synthesized AM1 was added to an E. coli cell lysate supernatant containing MBP, representing the polypeptide fusion partner for the AM1. This mimicked a cell lysate supernatant after homogenisation (cell disruption), ceramic filtration (volume reduction) and a cleavage step to release AM1 from its MBP fusion partner.

### Material and Methods

### Cell Cultures

For the precipitation experiments with ethanol, a cell lysate of an Escherichia coli (E. coli) culture with soluble expression product was produced.

E. coli Culture with Soluble Expression Product

The plasmid pRK793 (Kapust et al. 2001, Protein Engineering 14(12):993-1000) encoding the catalytic domain of tobacco etch virus (TEV) protease in the form of an MBP (maltose-binding protein) fusion protein that cleaves itself in vivo to yield a TEV protease catalytic domain with an N-terminal His-tag and a C-terminal polyarginine tag was provided by Dr. David S. Waugh (National Cancer Institute at Frederick, USA). The E. coli strain BL21(DE3)-RIL was used for protein expression.

For protein expression, the plasmid pRK793 was transformed into E. coli BL21(DE3)-RIL and grown in LB media containing 50 μg/mL Ampicillin and 17 μg/mL Chloramphenicol (antibiotics purchased from Sigma), at 37° C. with orbital shaking at 110 rpm. At mid-log phase (A₆₀₀=0.7) and the cells were induced to express with 0.4 mM IPTG (Quantum Scientific, Australia) for 3 h.

The cells were harvested by centrifugation (Sorvall Super T21 centrifuge) and the cell pellet was resuspended to an OD of 50 in a medium based on M9-medium. The medium contained 3 g Na₂HPO₄, 1.5 g KH₂PO₄, 0.5 g NaCl, 1 g NH₄Cl per litre MilliQ-filtered water and 100 mM MgSO₄ (All chemicals were purchased from Univar.) The pH of the medium was adjusted to pH 7. This medium mimicked a broth after fermentation with a minimal medium.

The cells were passed twice at 1000 bar in a Niro-Soavi (Italy, type NS1001L2K, S.N. 5817) high pressure homogenizor. The lysate was centrifuged at 4300 rpm for 30 min to remove the cell debris and unlysed cells. The supernatant containing MBP was filled into 50 mL Falcon tubes and stored at −20° C.

For the precipitation with ethanol the cell lysate supernatant was spiked with chemically synthesized AM1 peptide to a final concentration of 0.286 μg AM1 per μL. AM1 was custom synthesized by Genscript (Piscataway, USA). The purity was >95% by reversed-phase high performance liquid chromatography. The peptide was stored in a lyophilized form and was dissolved in 0.1% v/v TFA prior to use (c_{Am1}=1 mg/mL).

### Ethanol Precipitation

For the ethanol precipitation experiment, 420 μL of AM1 (in 0.1% v/v TFA, 1 μg/μL) was added to 1050 μL of lysate supernatant and mixed by vortexing. The samples were prepared with different ethanol concentrations ranging from 50-75% (v/v).

The samples were incubated on a roller mixer at speed adjustment 7.5 for 17 h. The tubes were centrifuged at 15,000 rpm for 10 min. The supernatants were is separated from the precipitates (pellets). The pellets were dried and redissolved in 400 μL 8M urea solution (in 0.1M Tris; pH=8). The protein present in the urea dissolved pellets, as well as the supernants was directly analyzed by Bradford and SDS-PAGE.

Peptide Analysis with RP-HPLC

For quantitative analysis of AM1 peptide in the supernatants and pellets, reversed-phase high performance liquid chromatography (RP-HPLC) was used with an analytical C18 column (Jupiter 00F-4053-E0, Phenomenex, UK). Mobile phase A was MQ-H₂O containing 0.1% v/v TFA, while phase B contained MQ-H₂O, 90% v/v acetonitrile (Lab-Scan, Irland) and 0.1% v/v TFA. The elution gradient increased from 35% B to 55% B over 20 min at a flow rate of 0.5 mL/min. The sample injection volume was 60 μL and the order of sample injection and measurement (supernatants and pellets) was randomized.

The retention time of AM1 at the above mentioned elution gradient and flow rate was t_{R}=21.5 min. To determine the amount of AM1 in the supernatants and pellets with RP-HPLC, a calibration curve was generated. Different amounts of AM1 (0.1-2 μg) were injected into the C18 column. The following equation was used to determine the amount of AM1 in the supernatants and pellets as a function of the peak area detected by RP-HPLC: Detected peak area/1207925=μg of AM1 peptide.

### Protein Analysis

Protein concentrations were determined with a standard Bradford assay. For qualitative protein analysis SDS-PAGE was conducted.

### Bradford Assay

Protein concentrations were determined using a Bio-Rad Protein Assay Kit (Bio-Rad, USA) using bovine serum albumin (BSA) as standard according to manufacturers instructions. Samples were measured in triplicates. For measuring the adsorption at 595 nm wavelength the UV-visible spectrophotometer UV-2450 from Shimadzu was used.

### SDS-PAGE

SDS-PAGE was preformed on 14% v/v Tris-glycine gels with a Bio-Rad Mini PROTEAN 3 Cell (Bio-Rad, USA). Electrophoresis was conducted at a constant voltage of 200V for 50-60 min in NuPAGE MES SDS Running Buffer (Invitrogen, UK). The gel was then recovered and the protein bands were stained by Coomassie brilliant blue R-250 for 45 min and then destained in a 70% v/v methanol, 10% v/v acetic acid solution for 30-45 min. The molecular weight markers used were Precision Plus Protein Standards 10-250 kDa (Bio-Rad, USA) and SDS-PAGE standards, Broad Range 6.5-200 kDa (Bio-Rad, USA).

### Results and Discussion

Purification of AM1 from Cell Lysate Containing MBP

The purification of soluble AM1 peptide from a cell lysate which also contained soluble maltose-binding protein (MBP), was studied. Since an E. coli strain which expresses AM1 fused to MBP, was not available, a model to investigate the purification of AM1 after it has been cleaved off from the fusion partner MBP was designed. In this model, chemically synthesized AM1 was added to a cell lysate containing MBP. This mimicked the later stage of purification, when AM1 peptide is released into the cell lysate after a homogenisation (cell disruption), ceramic filtration (volume reduction) and cleavage step (where AM1 is cleaved from the MBP-AM1 fusion protein by tobacco etch virus protease).

### Ethanol Precipitation

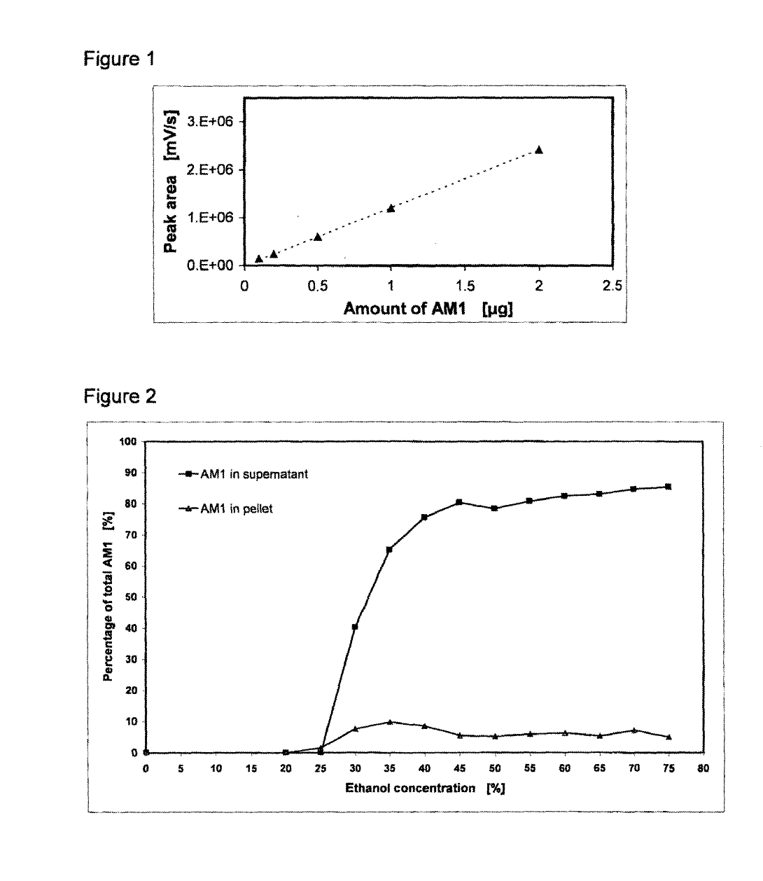

In FIG. 1, the percentage of total AM1 in the supernants and pellets, as a percentage of the amount of peptide in the starting material (AM1 spiked cell lysate), is plotted against the ethanol concentration. At ethanol concentrations of 50-75% (v/v), 80% to 85% of the total AM1 is detected in the supernatant, with approximately 5% of the AM1 being present in the precipitate. The percentage of total protein in the supernatants and pellets is plotted against the ethanol concentration in FIG. 1. The higher the ethanol concentration is, the more protein precipitates out of solution and is found in the pellet. These results were also confirmed by SDS-PAGE analysis (data not shown).

Summarizing the results: to purify AM1 from a cell lysate, a precipitation at greater than 65% (v/v) ethanol results in the majority of the protein in the cell lysate being precipitated whilst the peptide remains in solution. Over 80% of the peptide in the original lysate could be recovered from the supernatant. Accordingly, this method provides both high yields and high purity.

### Example 2

### Purification of P₁₁-2 Peptide, with Fusion Protein Cleavage in Cell Homogenate

We have modelled a further recombinant peptide production process that circumvents the need for chromatography, and the experimental results are described below. This process employs a small expression partner (12 kDa), Thioredoxin (Trx), fused to the C-terminal P₁₁-2 peptide, separated by the highly specific TEV protease consensus sequence. The peptide was cloned flush against the TEVp consensus sequence, such that the amino acid immediately preceding the TEVp cleavage site (the P′ position of the consensus sequence) is occupied by the first residue of the P₁₁-2 peptide. This results in the release of peptide with a native N-terminus. This is an important and deliberate aspect of our process since peptide function has been shown to be very sensitive to even minor sequence modifications. Rather than employ an initial purification step, the highly expressed fusion protein was enzymatically cleaved with recombinant TEVp, in the homogenate. The peptide was then isolated by a simple solvent precipitation step and its activity demonstrated by the formation of fibrils in solution.

### Materials and Methods

### Cloning

The expression vector, pET-Trx-P₁₁-2, was generated by inserting a cassette containing a spacer, TEVp consensus sequence and P₁₁-2 peptide sequence downstream of the Trx gene sequence in the pET48(b) (Novagen Merck Biosciences, Darmstadt, Germany) plasmid. The cassette was inserted via the Msc1 restriction site and was comprised of the following annealed, phosphorylated oligos (5′-3′):

Forward-
(SEQ ID NO: 2)
TCAGACCAACTCTATTACCTCCCTGTATAAATCTGCGGGCAGCGAAAACC
TGTACTTCCAACAGCAACGCTTCCAGTGGCAATTTGAACAGCAATAA
Reverse-
(SEQ ID NO: 3)
TTATTGCTGTTCAAATTGCCACTGGAAGCGTTGCTGTTGGAAGTACAGGT
TTTCGCTGCCCGCAGATTTATACAGGGAGGTAATAGAGTTGGTCTGA.

These oligos were designed based on codon usage preferences of E. coli, and correspond to a spacer sequence (QTNSITSLYKSAGS) (SEQ ID NO: 4), the modified TEVp consensus sequence (A P′ position residue) (ENLYFQ) (SEQ ID NO: 5), and the P₁₁-2 sequence (QQRFQWQFEQQ) (SEQ ID NO: 6), followed by a STOP codon. The expression vector was generated by the Protein Expression Facility (ARC SRC for Functional and Applied Genomics), University of Queensland.

### Cultivation Conditions and Mechanical Disruption

Recombinant E. coli BL21(DE3) (Novagen Merck Biosciences, Darmstadt, Germany) were cultivated in 2 L shake flasks in Luria Bertani (LB) media (1% w/v bacto-tryptone, 0.5% w/v yeast extract, 0.5% w/v NaCl) containing 50 mg L⁻¹ kanamycin. Overnight cultures were generated by inoculating 20 ml of LB media with a single colony and growing overnight at 37° C. to produce OD₆₀₀ values of approximately 2-3. These were diluted 1:100 in 400 ml of LB media and grown at 37° C. with 200 rpm shaking, before inducing at OD₆₀₀ 0.9 with 0.4 mM IPTG (Sigma Aldrich, St Louis, USA) for 3 h. Cells were harvested by centrifugation (4000×g/20 min) and pellets washed once with ice-cold 0.9% (w/v) NaCl and stored at −80° C. Cell pellets were resuspended in ice-cold TE buffer (10 mM Tris-Cl, 0.5 mM EDTA—pH 8.0 if the homogenate was for cleavage reactions or pH 7.2 if for purification), before homogenizing by a single passage at 1000 bar using a Panda 2K high pressure homogenizer (Niro Soavi, Parma, Italy). Homogenate was quantified by Bradford assay (BioRad) and aliquots were stored at −80° C.

### TEVp Cleavage of Trx-P₁₁-2 in Homogenate

All cleavage reactions were performed with recombinant TEVp, using homogenate prepared as above, where the Trx-P₁₁-2 content had been HPLC quantified. Cleavage reactions were performed in TEVp reaction buffer, at room temperature, in sealed tubes that had been purged of air with nitrogen. All reactions were stopped by the addition of equal volumes of either 6 M guanidine hydrochloride or 2×SDS-PAGE sample buffer and analyzed by HPLC (to quantify peptide generation) and SDS-PAGE (to confirm cleavage of the fusion protein), respectively.

Homogenate was heat-treated by incubating samples in a water bath at 55° C. for 15 min. The effect on the solubility of the fusion protein was assessed by SDS-PAGE and HPLC analysis of heat-treated homogenate before and after centrifugation (10000×g, 10 min). The effect on the stability of the P₁₁-2 peptide was analyzed by comparing cleavage reactions (at 0, 1, 2 and 3 h) with a final substrate concentration of 160 μM Trx-P₁₁-2 and 8 μM TEVp, prepared with heat-treated and non heat-treated homogenate. Conditions were optimized by evaluating reactions prepared with 8 μM TEVp and 160, 240 and 320 μM Trx-P₁₁-2 after 0, 1, 2 and 3 h. The effect of salt on enzyme activity was assessed by analyzing cleavage reactions prepared with 8 μM TEVp and 320 μM Trx-P₁₁-2, that were supplemented with either 50, 150 or 250 mM NaCl. These cleavage reactions were analyzed at 0, 1, 2 and 3 h by SDS-PAGE and HPLC. After 3 h, ethanol was added to a final concentration of 75% (v/v) and samples were incubated at room temperature for 10 min before centrifuging at 10 000×g for 5 min. The supernatants were diluted with water to reduce the percentage of ethanol and analyzed by HPLC.

### Precipitation Study

All precipitation experiments were performed with the same batch of Trx-P₁₁-2, cleaved with a 40:1 (substrate:enzyme) molar ratio, a substrate concentration of 320 μM (5.12 mg ml⁻¹) and in the presence of 250 mM NaCl. The precipitation samples were centrifuged for 5 min at 10000×g, and the supernatants collected and analyzed by HPLC to quantify the peptide present. The pellets were resuspended in 6 M guanidine hydrochloride to the same volume as the supernatants, and analysed by HPLC. Acetone and ethanol precipitations were performed using HPLC grade ethanol and acetone, which was added to samples to final concentrations of 20, 40, 60, 65, 70, 75, 80, and 85% (v/v) before incubating for 10 min at room temperature and centrifuging for 5 min at 10000×g. The supernatants were collected and dried using an RVC 2-18 speed-vac (Christ, Osterode, Germany), the pellets were air-dried and both were resuspended in 6 M guanidine hydrochloride. The samples were analyzed by HPLC to quantify the peptide and Bradford assay to quantify the total protein remaining. The peptide and protein remaining in the pellets and supernatants were compared to the results obtained for the starting cleavage reactions to determine the percentages of peptide and protein in each fraction.

### Preparation of P₁₁-2 Peptide

Cell pellets from Trx-P₁₁-2 culture were thawed and homogenized in TEVp reaction buffer, and the fresh homogenate immediately heat-treated at 55° C. for 15 min. The heat-treated homogenate was added directly to a cleavage reaction with 8 μM TEVp and 320 μM Trx-P₁₁-2, containing 250 mM NaCl. After 3 hours, the cleavage reaction was precipitated with 75% (v/v) ethanol for 10 min, centrifuged (10 000×g/5 min) and the resulting supernatant was dried by speedvac. The peptide preparation was resuspended in water for analysis by HPLC and mass spectrometry.

### Reversed-Phase HPLC Purification and Quantitation

Reversed-phase HPLC(RP-HPLC) was carried out using an analytical C5 column (Jupiter, 150 mm×4.6 mm, 5μ, 300 Å) (Phenomenex, Torrance, Calif., USA) connected to a LC-10Avp series HPLC system (Shimadzu, Kyoto, Japan) with a mobile phase A, 0.1% (v/v) trifluoroacetic acid (TFA) in water and B, 90% (v/v) acetonitrile, 0.1% (v/v) TFA in water. Initial analysis of fusion protein and cleavage was performed using a linear gradient from 10% B to 60% B in 50 min at a flow rate of 1 ml min⁻¹. Peptides were analyzed, collected and quantified using a linear gradient from 10% B to 35% B in 25 min. Final peptide preparation was assessed using a linear gradient from 10% B to 90% B in 80 min. All samples were diluted 1:2 in 6 M guanidine-HCl prior to injection for the purpose of column preservation unless stated otherwise.

### Mass Spectrometry

Peak fractions were air-dried under nitrogen. ESI-Q-TOF MS of peptides was carried out on an API QSTAR Pulsar Hybrid Quadrupole TOF LS/MS/MS Mass Spectrometer (Applied Biosystems/MDS Sciex, Foster City, Calif., USA). AnalystQS1.1 was used for data acquisition and processing.

### SDS-PAGE

Samples were analyzed by SDS-PAGE with 14% (w/v) Tris-glycine gels. Samples were prepared with 2× sample buffer (200 mM Tris-Cl, 30% glycerol, 5% SDS, 0.1% (w/v) Coomassie brilliant blue R250, 100 mM DTT). Electrophoresis was performed using a Bio-Rad Mini-Protean III system (Bio-Rad, CA, USA) at a constant voltage of 100 V for 1 h, and gels were stained with Coomassie brilliant blue R250. Bio-Rad Broad-range Unstained Molecular Weight Standard (catalogue # 161-0317) was used.

### Protein Estimation

Total protein was quantified by Bradford Assay (Bio-Rad, CA, USA) according to the manufacturer's instructions. Relative percentage of target protein was estimated by Bioanalyzer chip (Agilent, CA, USA) and average expression levels were calculated across consecutive batches of shake flask expression. Purified Trx-P₁₁-2 and TEVp were quantified by Bioanalyzer and via A₂₈₀ measurement m using theoretical molar extinction coefficients as calculated using ProtParam (EXPASY Proteomics Server) of 22585 M⁻¹ cm⁻¹ and 32220 M⁻¹ cm⁻¹ respectively. Pure peptide collected from RP-HPLC was initially quantified via A₂₈₀ measurement using a theoretical molar extinction coefficient of 5500 M⁻¹ cm⁻¹, calculated using ProtParam (EXPASY Proteomics Server) for the P₁₁-2 sequence. Quantified Trx-P₁₁-2 fusion protein and P₁₁-2 peptide was used to generate HPLC standard curves using the Class VP 7.4 software and all subsequent measurements were made by RP-HPLC.

### Results

### Cloning and Expression of Trx-P₁₁-2

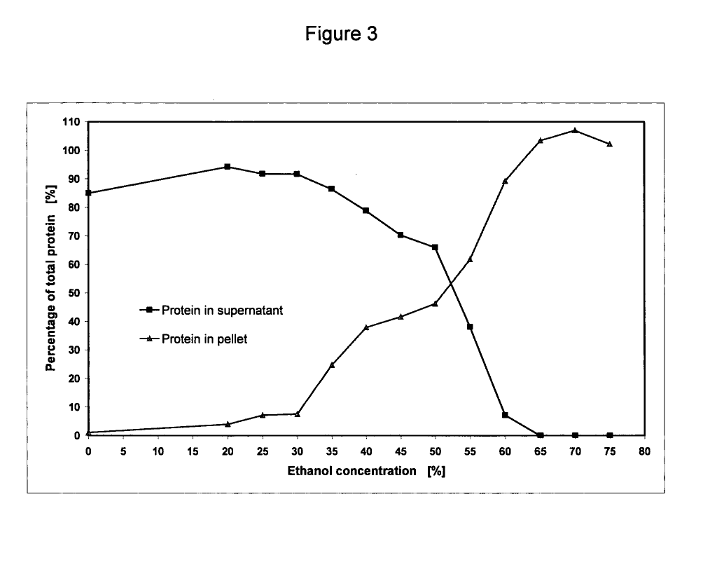

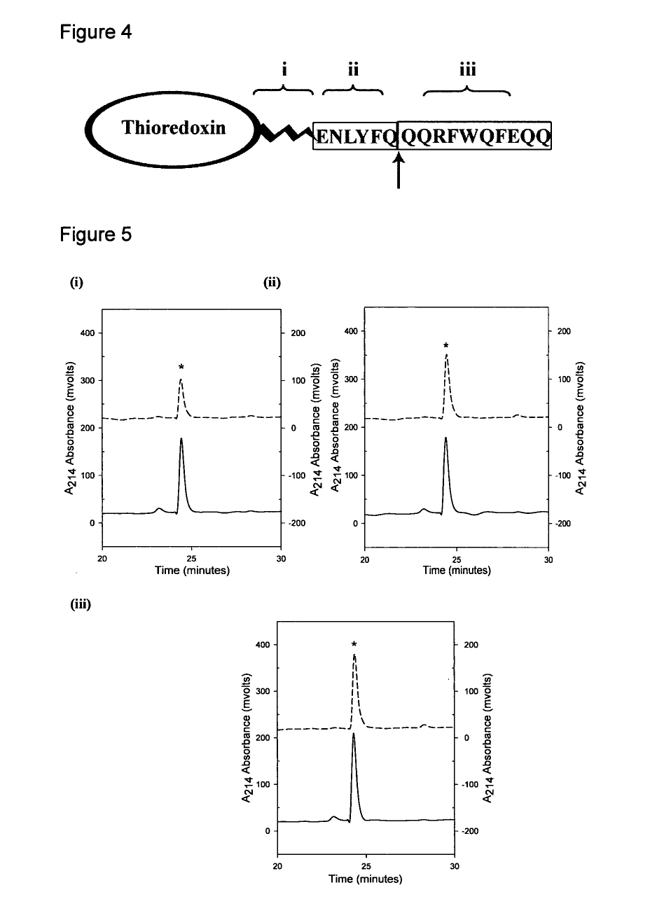

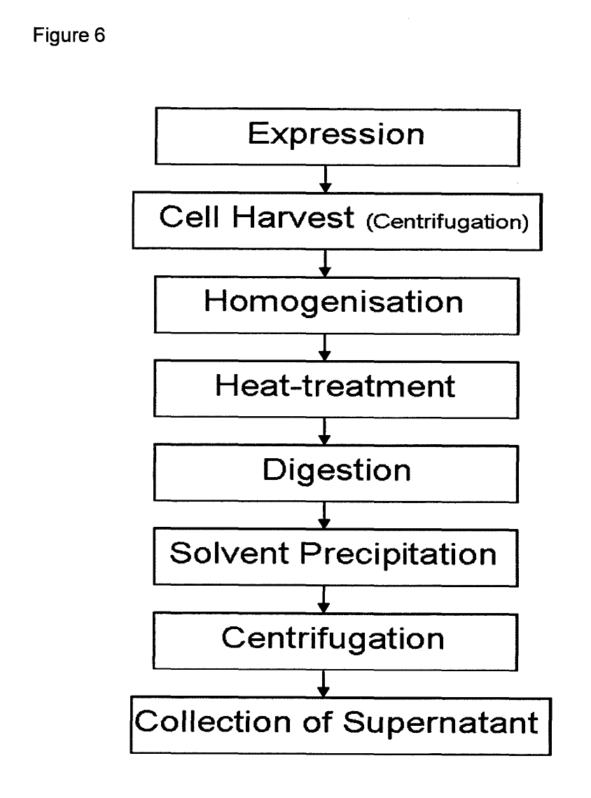

The design of the Trx-P₁₁-2 expression product is shown in FIG. 2. The P₁₁-2 peptide is positioned immediately C-terminal of the TEVp cleavage site, such that the native P′ position of the TEVp consensus sequence (Gly) is not occupied by Gly but rather by the first residue of the P₁₁-2 peptide (Gln). This region is then separated from the fusion protein by a spacer sequence. The expected molecular weight for the fusion protein is 15.9 kDa while the peptide is 1.55 kDa, indicating the fusion protein is comprised of 10% target peptide. The construct was expressed in E. coli BL21(DE3) cells with LB media in shake flasks and the cells routinely reached OD₆₀₀ levels of approximately 3 after 3 h induction. SDS-PAGE confirmed the presence of the expression product of Trx-P₁₁-2 at the expected molecular weight of approximately 16 kDa (data, not shown). After homogenization of induced cells, Bradford analysis indicated that the total cell protein (TCP) was approximately 560 mg per litre of culture. The fusion protein constituted approximately 60% of TCP, corresponding to 330 mg of Trx-P₁₁-2 per litre.

### Peptide Purification by Selective Precipitation of Contaminant Polypeptides

Isolation of the peptide after TEVp cleavage of Trx-P₁₁-2 in homogenate was attempted with selective precipitation of contaminating proteins using acetone and ethanol. Initial solvent precipitation trials indicated that, in standard TEVp reaction buffer (10 mM Tris-Cl, 0.5 mM EDTA, 5 mM DTT, pH 8.0), P₁₁-2 peptide co-precipitated with the contaminating proteins in the homogenate. This was in contrast to purified peptide, which was found to be soluble at high solvent concentrations (results not shown). Cleavage of fusion protein with TEVp in the presence of 50 mM, 150 mM and 250 mM sodium chloride was tested and the results indicated that addition of salt did not have an effect on TEVp cleavage, (data not shown). HPLC analysis demonstrated the solubility of the peptide with the addition of increasing amounts of salt. The yield of peptide in the supernatants after precipitations performed in the presence of 50 mM, 150 mM and 250 mM salt was 55%, 81% and 95%, respectively (data not shown).

The effectiveness of precipitation in the presence of 250 mM salt, as a means of isolating P₁₁-2 peptide from homogenate after TEVp cleavage, was further investigated using acetone and ethanol. Preliminary studies indicated that precipitation using ethanol and acetone was complete in less than 5 minutes. (data not shown). Both acetone and ethanol were effective at precipitating over 90% of the contaminating proteins at concentrations of >65% (v/v). FIG. 3 (a) shows that acetone precipitation resulted in peptide losses exceeding 30% when 75% (v/v) acetone was used, with the peptide being almost completely insoluble at 85% (v/v) acetone. However, ethanol precipitation at 75% (v/v) resulted in peptide yields of more than 90% with less than 3% of contaminating protein remaining in the supernatant with the peptide (see FIG. 3 (b)). Significant losses of peptide (>70%) occurred when the ethanol concentration was raised to 85% (v/v).

### Preparation of P₁₁-2 Peptide

The finalized peptide production process was assessed using 1 L of Trx-P₁₁-2 culture (at OD₆₀₀ 3.3) which yielded 28 ml of homogenate at 20 mg/ml TCP, consisting of 12 mg/ml Trx-P₁₁-2 fusion protein. After heat-treatment, and 3 hours of cleavage with a substrate:enzyme molar ratio of 40:1, containing 250 mM NaCl, the reaction was approximately 45% complete (data not shown). The contaminating proteins were separated from the peptide by precipitation with 75% (v/v) ethanol and the concentrated supernatant was analyzed by HPLC and mass spectrometry (FIG. 12). A peak corresponding to approximately 6 μg of peptide eluted at the expected time (24 min), with only minimal background peaks. FIG. 4 shows that mass spectrometry confirmed the peak was comprised of P₁₁-2 peptide, with an expected molecular weight of 1551 Da. The mass spectrum also further demonstrated the purity of the peptide preparation due to the absence of major background peaks. The peptide yield for each step was 95% for Trx-P₁₁-2 after heat-treatment, 45% after cleavage and 95% yield of P₁₁-2 peptide after removal of contaminating proteins with solvent precipitation. This gave a cumulative yield of 40% and correlated to the production of approximately 10 mg of P₁₁-2 peptide per 560 mg of TCP, for the process described.

### Discussion

The optimized process design described above relies on the fusion of the target peptide P₁₁-2 to a small carrier protein, Thioredoxin (Trx), with cleavage by tobacco Tobacco Etch Virus Protease (TEVp). Practical realisation of this construct has been achieved in the current study. Although the most common native consensus sequence for TEVp is ENLYFQG, with cleavage occurring after the Gln residue, research has shown there is no absolute requirement for a Gly residue at the position following the cleavage site (the P′ position). We therefore cloned the P₁₁-2 sequence immediately after the TEVp cleavage site to allow generation of peptide with a native N-terminus and maintain peptide functionality. We also included a spacer arm between the Trx fusion partner and the cleavage site to help minimize steric hindrance that may affect TEVp cleavage. Trx was chosen as the expression partner as it is highly soluble and stable, and its small size allows for a desirable ratio between fusion partner and peptide at approximately 10:1 (process economics are heavily influenced by the choice of fusion partner).

The fusion protein was expressed soluble in E. coli BL21(DE3) cells and comprised up to 60% of Total Cell Protein (TCP). As expected, this level was achieved in part due to the down-regulation of host-cell house-keepings genes as a consequence of the metabolic burden placed on the cells by the recombinant expression. This high level of expression obtained in shake flasks is not unusual in BL21(DE3) cells, with reports of expression in excess of 70% of TCP for small recombinant proteins. When judged against other reports for Trx-peptide fusion proteins expressed in shake flasks, that range from 15%, 30% to 50%, the result is acceptable. This also proves that the peptide, in fusion with Trx, was not toxic to the cells, which is significant because the P₁₁-2 peptide exhibits antimicrobial behaviour.

The most important requirement for realizing this process was the removal of chromatography steps. In order to achieve this, feasibility of TEVp cleavage without initially isolating the fusion protein from the homogenate via IEXC was assessed. Preliminary experiments indicated that, while reasonable levels of cleavage could be achieved in homogenate, the peptide was being quickly degraded, most likely by cellular proteases. However, the fusion protein was very stable in the homogenate and peptide degradation products only appeared after cleavage by TEVp. This protease activity was eliminated by a heat-treatment step of 55° C. for 15 min prior to TEVp cleavage. Although heat treatment is commonly used to inactivate proteases, using it in conjunction with a highly soluble, heat-stable fusion tag, Trx, meant there was no loss of target protein due to heat-induced precipitation.

Proteolytic cleavage trials demonstrated that cleavage efficiency could be markedly improved without the need for additional enzyme by increasing the substrate concentration, which is in accord with Michaelis-Menton enzyme kinetics. After optimisation of reaction conditions, it was found that 50% cleavage could be achieved in homogenate using a 40:1 (substrate:enzyme) molar ratio with a substrate concentration of 5.12 mg/ml (320 μM). This corresponded to approximately half the enzyme efficiency achieved for purified substrate, which was a very positive result.

There was also a need to remove the chromatography steps traditionally associated with peptide isolation to achieve the modelled costings. In Example 1, we had shown that solvent precipitation is an effective method of isolating peptides from high molecular weight contaminants, since the lack of tertiary structure in the peptide allows it to maintain solubility while most other proteins undergo denaturation and aggregation. This step was applied to here to remove the contaminating proteins from the P₁₁-2 peptide after TEVp cleavage in homogenate. However, in the absence of salt, P₁₁-2 peptide was interacting with homogenate proteins and co-precipitating after solvent addition. This interaction was negated by the addition of 250 mM salt in the TEVp reaction buffer, indicating the association was most likely ionic in nature. Tests confirmed that precipitation at room temperature with either 70-75% (v/v) ethanol or acetone were effective means of producing peptide at over 97% purity. The losses suffered were minimal (<5%) and comparable to what would be expected using RP-HPLC. This is a very effective method of isolating peptide without chromatography or irreversible volume increases, as ethanol is volatile and could be recycled in larger processes. In addition, the solvent step should have no effect on peptide function since the structural integrity of P₁₁-2 and other beta-sheet forming peptides has been shown to be conserved in polar organic solvents such as ethanol and acetone.

The final process for production of P₁₁-2 peptide is presented in FIG. 5. Despite the apparent low yield of peptide at 10 mg per litre of fermentation broth, this is a significant result due to the simplicity of the process. It also compares very well with other conventional recombinant peptide production methods that traditionally employ multiple chromatography steps and have yields that range from 1-50 mg of peptide per litre of fermentation broth. The native and functional peptide was obtained at an overall process yield of 40% at high purity, without the process expense imposed by chromatography. It also required only limited centrifugation and utilised a very cheap and recyclable solvent, ethanol, to isolate the peptide. Moreover, if fermentation was scaled to a bioreactor where BL21(DE3) cells routinely reach cell densities ranging from 60-100 g/L of dry cell weight, even in minimal media, the centrifugation step associated with cell harvest may be unnecessary as the culture could be homogenized directly (Middelberg et al 1991). Such high cell densities represent TCP levels in excess of 50 g/L, which is 100-fold higher than the levels achieved with our shake-flask expressions. Thus, if the scale-up of this process proved to be linear, gram quantities of peptide could be produced per litre of fermentation broth.

Modelling suggests that this process, if further optimized and scaled-up to incorporate ceramic filtration and vacuum distillation, could potentially produce recombinant peptide, on a large scale, at a cost of less than $5 per g. This is below the current costings associated with large-scale, synthetic peptide production (Marx 2005, Chemical and Engineering News 83(11)). Expenditure could be reduced significantly if the recombinant production of TEVp was more efficient, as it is one of the major contributor to the cost of the process described here. We believe this work is an important step towards economically viable recombinant production of peptides, as it represents a novel method for the commercial production of peptide specifically, rather than simply adapting established methods for recombinant protein production. Subsequently, this innovative, cost-effective process brings us closer to making peptide-based biomaterials a commercially feasible replacement for current polymer-based products.

The various features and embodiments of the present invention, referred to in individual sections above apply, as appropriate, to other sections, mutatis mutandis. Consequently features specified in one section may be combined with features specified in other sections as appropriate.

All publications mentioned in the above-specification are herein incorporated by reference. Various modifications and variations of the described methods and products of the invention will be apparent to those of skill in the art without departing from the spirit and scope of the invention. Although the invention has been described in connection with specific preferred embodiments, it should be understood that that the invention as claimed should not be unduly limited to such specific embodiments. Indeed, in various modifications of the described modes for carrying out the invention which are apparent to those skilled in the relevant fields are intended to be within the scope of the following claims.

1-26. (canceled)

27. A method of purifying a recombinant peptide which method comprises:
providing a solution comprising a recombinant peptide and a polypeptide fusion partner;
(ii) adding a non-aqueous polar solvent to the solution to a concentration of from 10% v/v to 80% v/v thereby selectively precipitating proteins present in the solution and thereby retaining the recombinant peptide in solution; and
(iii) separating the resulting precipitate to obtain a solution comprising the recombinant peptide.

28. The method of claim 27, wherein the solution comprising a recombinant peptide and a polypeptide fusion partner is a cell lysate or a partially purified derivative of a cell lysate.

29. The method of claim 27, wherein the recombinant peptide has been secreted extracellularly.

30. The method of claim 27, wherein the non-aqueous polar solvent is added to a concentration of from 30% v/v to 80% v/v.

31. The method of claim 27, wherein the non-aqueous polar solvent is added to a concentration of from 50% v/v to 80% v/v.

32. The method of claim 27, wherein the non-aqueous polar solvent is added to a concentration of from 65% v/v to 80% v/v.

33. The method of claim 27, wherein the non-aqueous polar solvent is an aliphatic alcohol.

34. The method of claim 27, wherein the non-aqueous polar solvent is a polar aprotic solvent.

35. The method of claim 27, wherein the recombinant peptide comprises from 10 to 70 amino acids.

36. The method of claim 27, wherein the recombinant peptide comprises from 10 to 30 amino acids.

37. The method of claim 27, wherein the fusion partner is a protein that enhances solubility and/or stability of the recombinant peptide.

38. The method of claim 27, wherein the fusion partner comprises at least 70 amino acids.

39. The method of claim 27, wherein the recombinant peptide has been expressed as a fusion polypeptide with the fusion partner in a microbial host.

40. The method of claim 27, wherein the recombinant peptide has been expressed as a fusion polypeptide with the fusion partner using a fungal host.

41. The method of claim 27, wherein, in step (i), the solution comprising a recombinant peptide and a polypeptide fusion partner further comprises a cleavage agent which cleaves the recombinant polypeptide and the fusion partner.

42. The method of claim 41, wherein the cleavage agent is a protease.

43. The method of claim 41, wherein the solution comprising a recombinant peptide and a polypeptide fusion partner has been heated to inactivate host cell proteins prior to cleavage of the fusion polypeptide.

44. The method of claim 43, wherein the solution comprising a recombinant peptide and a polypeptide fusion partner has been heated to a temperature not more than 70° C. to inactivate host cell proteins.

45. The method of claim 44, wherein the solution comprising a recombinant peptide and a polypeptide fusion partner has been heated to a temperature not less than 45° C. to inactivate host cell proteins.

46. The method of claim 27, wherein the solution comprising a recombinant peptide and a polypeptide fusion partner is a cell lysate supernatant.

47. The method of claim 27, wherein in step (ii) the precipitation step takes places at a temperature of from 10° C. to 37° C.

48. The method of claim 27, wherein the conditions include the presence of at least 100 mM salt.

49. The method of claim 27, further comprising the step (iv) of recovering the recombinant peptide from the solution obtained in step (iii).

50. The method of claim 27, with the proviso that the fusion partner is not carbonic anhydrase or a fragment thereof.

51. A recombinant peptide obtained by the method of claim 27.
